(19) United States
(12) Patent Application Publication
(10) Pub. No.: US 2009/0036842 A1
(43) Pub. Date: Feb. 5, 2009

(54) Consumable Activation Lever For Injection Device

(76) Inventor: Raffi Pinedjian, Fountain Valley, CA (US)

Correspondence Address: ALCON, IP LEGAL, TB4-8, 6201 SOUTH FREEWAY, FORT WORTH, TX, 76134, US

(21) Appl. No.: 11/833,369

(22) Filed: Aug. 3, 2007

(51) Int. Cl. A61M 5/315

(52) U.S. Cl. 604/272

(57) ABSTRACT

A disposable injection device assembly includes a dispensing chamber housing, a plunger, and a lever. The dispensing chamber housing is coupled to a needle and has an inner surface and an outer surface. The inner surface partially defines a dispensing chamber for holding a quantity of a substance. The plunger is engaged with the inner surface of the dispensing chamber housing, is capable of sliding in the dispensing chamber housing, and is fluidly sealed to the inner surface of the dispensing chamber housing. A housing at least partially surrounds the dispensing chamber housing. The lever is located on the housing. The lever actuates the injection device assembly.

### RELATED APPLICATIONS

This Application is a continuation-in-part of U.S. patent application Ser. No. 11/581,629 filed Oct. 16, 2006, U.S. patent application Ser. No. 11/581,630 filed Oct. 16, 2006, U.S. patent application Ser. No. 11/581,591 filed Oct. 16, 2006.

### BACKGROUND OF THE INVENTION

The present invention relates to a medical device and more particularly to an injection device or subassembly thereof with a consumable activation lever.

Several diseases and conditions of the posterior segment of the eye threaten vision. Age related macular degeneration (ARMD), choroidal neovascularization (CNV), retinopathies (e.g., diabetic retinopathy, vitreoretinopathy), retinitis (e.g., cytomegalovirus (CMV) retinitis), uveitis, macular edema, glaucoma, and neuropathies are several examples.

These, and other diseases, can be treated by injecting a drug into the eye. Such injections are typically manually made using a conventional syringe and needle. FIG. 1 is a perspective view of a prior art syringe used to inject drugs into the eye. In FIG. 1, the syringe includes a needle 105, a luer hub 110, a chamber 115, a plunger 120, a plunger shaft 125, and a thumb rest 130. As is commonly known, the drug to be injected is located in chamber 115. Pushing on the thumb rest 130 causes the plunger 120 to expel the drug through needle 105.

In using such a syringe, the surgeon is required to puncture the eye tissue with the needle, hold the syringe steady, and actuate the syringe plunger (with or without the help of a nurse) to inject the fluid into the eye. The volume injected is typically not controlled in an accurate manner because the vernier on the syringe is not precise relative to the small injection volume. Fluid flow rates are uncontrolled. Reading the vernier is also subject to parallax error. Tissue damage may occur due to an “unsteady” injection. Reflux of the drug may also occur when the needle is removed from the eye.

An effort has been made to control the delivery of small amounts of liquids. A commercially available fluid dispenser is the ULTRA™ positive displacement dispenser available from EFD Inc. of Providence, R.I. The ULTRA dispenser is typically used in the dispensing of small volumes of industrial adhesives. It utilizes a conventional syringe and a custom dispensing tip. The syringe plunger is actuated using an electrical stepper motor and an actuating fluid. Parker Hannifin Corporation of Cleveland, Ohio distributes a small volume liquid dispenser for drug discovery applications made by Aurora Instruments LLC of San Diego, Calif. The Parker/Aurora dispenser utilizes a piezo-electric dispensing mechanism. Ypsomed, Inc. of Switzerland produces a line of injection pens and automated injectors primarily for the self-injection of insulin or hormones by a patient. This product line includes simple disposable pens and electronically-controlled motorized injectors.

U.S. Pat. No. 6,290,690 discloses an ophthalmic system for injecting a viscous fluid (e.g. silicone oil) into the eye while simultaneously aspirating a second viscous fluid (e.g. perflourocarbon liquid) from the eye in a fluid/fluid exchange during surgery to repair a retinal detachment or tear. The system includes a conventional syringe with a plunger. One end of the syringe is fluidly coupled to a source of pneumatic pressure that provides a constant pneumatic pressure to actuate the plunger. The other end of the syringe is fluidly coupled to an infusion cannula via tubing to deliver the viscous fluid to be injected.

It would be desirable to have a portable hand piece for reliably injecting a drug into the eye. Such a hand piece may have a reusable section and a disposable section. The reusable section may contain elements, such as a power supply and a controller, for operating the injection system. The disposable section may include the injection needle and other components that are appropriate only for a single use. In such a two piece system, the interface between the two pieces must function to properly execute an injection procedure. A switch on the disposable section should be mechanically linked to and communicate with the reusable section. Such a switch design would be a desirable feature of two piece injection system.

### SUMMARY OF THE INVENTION

In one embodiment consistent with the principles of the present invention, the present invention is a disposable injection device assembly including a dispensing chamber housing, a plunger, a lever, and a switch. The dispensing chamber housing is coupled to a needle and has an inner surface and an outer surface. The inner surface partially defines a dispensing chamber for holding a quantity of a substance. The plunger is engaged with the inner surface of the dispensing chamber housing, is capable of sliding in the dispensing chamber housing, and is fluidly sealed to the inner surface of the dispensing chamber housing. A housing at least partially surrounds the dispensing chamber housing. The lever is located on the housing. The switch is located adjacent to the lever. When the lever is depressed, the lever contacts the switch.

In another embodiment consistent with the principles of the present invention, the present invention is a disposable injection device assembly including a dispensing chamber housing, a plunger, and a lever. The dispensing chamber housing is coupled to a needle and has an inner surface and an outer surface. The inner surface partially defines a dispensing chamber for holding a quantity of a substance. The plunger is engaged with the inner surface of the dispensing chamber housing, is capable of sliding in the dispensing chamber housing, and is fluidly sealed to the inner surface of the dispensing chamber housing. A housing at least partially surrounds the dispensing chamber housing. The lever is located on the housing. The lever has a protrusion on one end. When the lever is depressed, the protrusion is moved.

It is to be understood that both the foregoing general description and the following detailed description are exemplary and explanatory only and are intended to provide further explanation of the invention as claimed. The following description, as well as the practice of the invention, set forth and suggest additional advantages and purposes of the invention.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 is a perspective view of a prior art syringe.

FIG. 2 is one view of an ophthalmic medical device including a disposable tip segment and a limited reuse assembly according to an embodiment of the present invention.

FIG. 3 is another embodiment of a limited reuse assembly according to the principles of the present invention.

FIG. 4 is a cross section view of a disposable tip segment and a limited reuse assembly according to an embodiment of the present invention.

FIGS. 5A-5B are cross section views of a tip segment for an ophthalmic medical device according to an embodiment of the present invention.

FIGS. 6A-6B are cross section views of a tip segment for an ophthalmic medical device according to an embodiment of the present invention.

FIGS. 7A-7C depict side and end views of the tip segment of FIGS. 5 and 6.

FIG. 8 is an cross section view of a tip segment for an ophthalmic medical device according to an embodiment of the present invention.

FIG. 9 is an cross section view of a tip segment for an ophthalmic medical device according to an embodiment of the present invention.

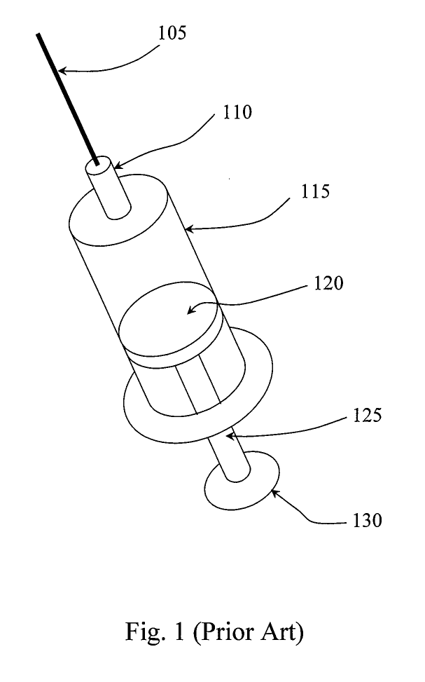

### DETAILED DESCRIPTION OF THE PREFERRED EMBODIMENTS

Reference is now made in detail to the exemplary embodiments of the invention, examples of which are illustrated in the accompanying drawings. Wherever possible, the same reference numbers are used throughout the drawings to refer to the same or like parts.

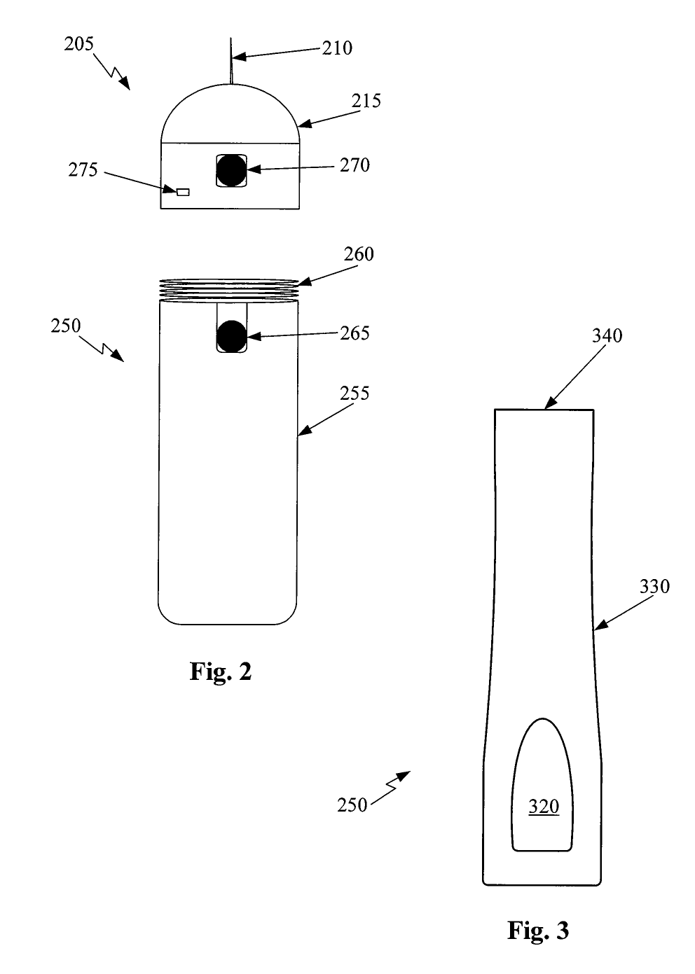

FIG. 2 is one view of an ophthalmic medical device including a disposable tip segment and a limited reuse assembly according to an embodiment of the present invention. In FIG. 2, the medical device includes a tip segment 205 and a limited reuse assembly 250. The tip segment 205 includes a needle 210, a housing 215, a button 270, and an optional light 275. The limited reuse assembly 250 includes a housing 255, a lock mechanism 265, and a threaded portion 260.

Tip segment 205 is capable of being connected to and removed from limited reuse assembly 250. In this embodiment, tip segment 205 has a threaded portion on an interior surface of housing 215 that screws onto the threaded portion 260 of limited reuse assembly 250. In addition, lock mechanism 265 secures tip segment 215 to limited reuse assembly 250. Lock mechanism 265 may be in the form of a button, a sliding switch, or a cantilevered mechanism. Other mechanisms for connecting tip segment 205 to limited reuse assembly 250, such as those involving structural features that mate with each other, are commonly known in the art and are within the scope of the present invention.

Needle 210 is adapted to deliver a substance, such as a drug, into an eye. Needle 210 may be of any commonly known configuration. Preferably, needle 210 is designed such that its thermal characteristics are conducive to the particular drug delivery application. For example, when a heated drug is to be delivered, needle 210 may be relatively short (several millimeters) in length to facilitate proper delivery of the drug.

Button 270 is adapted to provide an input to the system. For example, button 270 may be used to activate the system or to turn on a heater. Other switches, buttons, or user-directed control inputs are commonly known and may be employed with limited reuse assembly 250 and/or tip segment 205.

Optional light 275 is illuminated when tip segment 205 is ready to be used. Optional light 275 may protrude from housing 215, or it may be contained within housing 215, in which case, optional light 275 may be seen through a clear portion of housing 215. In other embodiments, optional light 275 may be replaced by an indicator, such as a liquid crystal display, segmented display, or other device that indicates a status or condition of disposable tip segment 205. For example, optional light 275 may also pulse on and off to indicate other states, such as, but not limited to a system error, fully charged battery, insufficiently charged battery or faulty connection between the tip segment 205 and limited use assembly 250. While shown on tip segment 205, optional light 275 or other indicator may be located on limited reuse assembly 250.

FIG. 3 is another embodiment of a limited reuse assembly according to the principles of the present invention. Limited reuse assembly 250 includes, a display 320, and a housing 330. Disposable tip segment 205 attaches to end 340 of limited reuse assembly 250. Display 320 is a liquid crystal display, segmented display, or other device that indicates a status or condition of disposable tip segment 205 or limited reuse assembly 250.

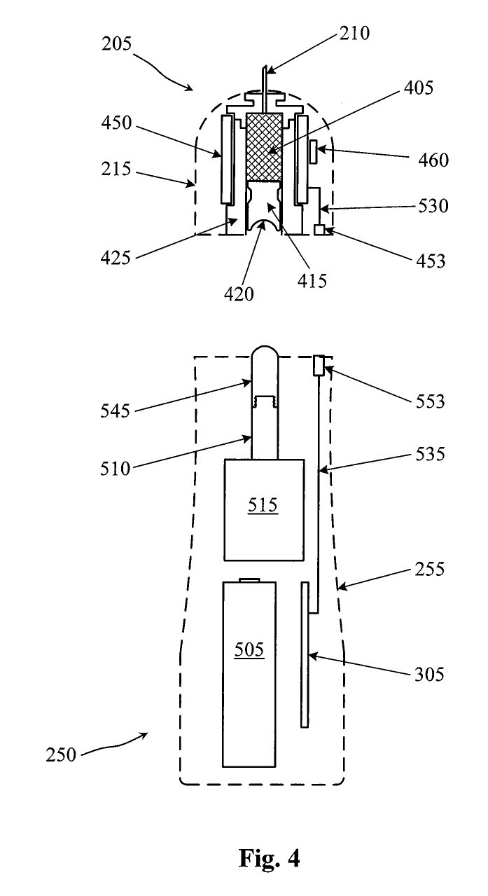

FIG. 4 is cross section view of a disposable tip segment and a limited reuse assembly according to an embodiment of the present invention. FIG. 4 generally shows how tip segment 205 interfaces with limited reuse assembly 250. In the embodiment of FIG. 4, tip segment 205 includes plunger interface 420, plunger 415, dispensing chamber housing 425, tip segment housing 215, temperature control device 450, thermal sensor 460, needle 210, dispensing chamber 405, interface 530, and tip interface connector 453. Limited reuse assembly 250 includes mechanical linkage interface 545, actuator shaft 510, actuator 515, power source 505, controller 305, limited reuse assembly housing 255, interface 535, and limited reuse assembly interface connector 553.

In tip segment 205, plunger interface 420 is located on one end of plunger 415. The other end of plunger 415 forms one end of dispensing chamber 405. Plunger 415 is adapted to slide within dispensing chamber 405. The outer surface of plunger 415 is fluidly sealed to the inner surface of dispensing chamber housing 425. Dispensing chamber housing 425 surrounds the dispensing chamber 405. Typically, dispensing chamber housing 425 has a cylindrical shape. As such, dispensing chamber 405 also has a cylindrical shape.

Needle 210 is fluidly coupled to dispensing chamber 405. In such a case, a substance contained in dispensing chamber 405 can pass through needle 210 and into an eye. Temperature control device 450 at least partially surrounds dispensing chamber housing 425. In this case, temperature control device 450 is adapted to heat and/or cool dispensing chamber housing 425 and any substance contained in dispensing chamber 405. Interface 530 connects temperature control device 450 with tip interface connector 453.

Optional thermal sensor 460 provides temperature information to assist in controlling the operation of temperature control device 450. Thermal sensor 460 may be located near dispensing chamber housing 425 and measure a temperature near dispensing chamber housing 425 or may be located in thermal contact with dispensing chamber housing 425, in which case it measures a temperature of dispensing chamber housing 425. Thermal sensor 460 may be any of a number of different devices that can provide temperature information. For example, thermal sensor 460 may be a thermocouple or a resistive device whose resistance varies with temperature. Thermal sensor is also electrically coupled to interface 530 or other similar interface.

The components of tip segment 205, including dispensing chamber housing 425, temperature control device 450, and plunger 415 are at least partially enclosed by tip segment housing 215. In one embodiment consistent with the principles of the present invention, plunger 415 is sealed to the interior surface of dispensing chamber housing 425. This seal prevents contamination of any substance contained in dispensing chamber 405. For medical purposes, such a seal is desirable. This seal can be located at any point on plunger 415 or dispensing chamber housing 425.

In limited reuse assembly 250, power source 505 provides power to actuator 515. An interface (not shown) between power source 505 and actuator 515 serves as a conduit for providing power to actuator 515. Actuator 515 is connected to actuator shaft 510. When actuator 515 is a stepper motor, actuator shaft 510 is integral with actuator 515. Mechanical linkage interface 545 is connected to actuator shaft 510. In this configuration, as actuator 515 moves actuator shaft 510 upward toward needle 210, mechanical linkage interface 545 also moves upward toward needle 210. In other embodiments of the present invention, mechanical linkage interface 545 and actuator shaft 510 are a single component. In other words, a shaft connected to actuator 515 includes both actuator shaft 510 and mechanical linkage interface 545 as a single assembly.

In limited reuse assembly 250, power source 505 is typically a rechargeable battery, such as a lithium ion battery, although other types of batteries may be employed. In addition, any other type of power cell is appropriate for power source 505. Power source 505 provides current to dispensing chamber housing 425 to heat it and change its shape. Optionally, power source 505 can be removed from housing 255 through a door or other similar feature (not shown).

Controller 305 is connected via interface 535 to limited reuse assembly interface connecter 553. Limited reuse assembly interface connecter 553 is located on a top surface of limited reuse assembly housing 255 adjacent to mechanical linkage interface 545. In this manner, both limited reuse assembly interface connector 553 and mechanical linkage interface 545 are adapted to be connected with tip interface connector 453 and plunger interface 420, respectively.

Controller 305 and actuator 515 are connected by an interface (not shown). This interface (not shown) allows controller 305 to control the operation of actuator 515. In addition, an interface between power source 505 and controller 305 allows controller 305 to control operation of power source 505. In such a case, controller 305 may control the charging and the discharging of power source 505 when power source 505 is a rechargeable battery.

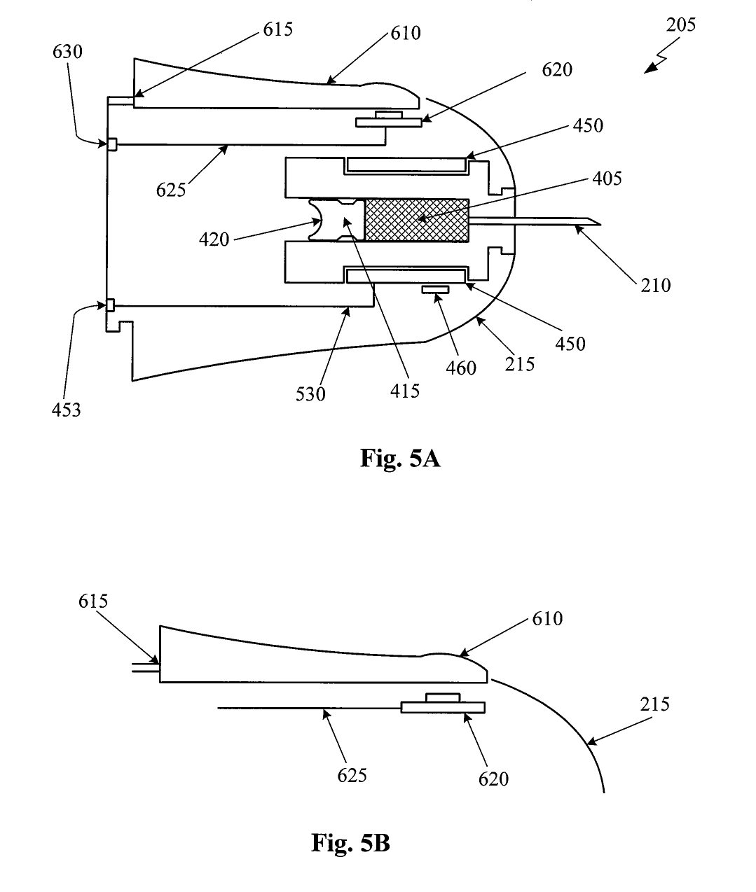

Controller 305 is typically an integrated circuit with power, input, and output pins capable of performing logic functions. In various embodiments, controller 305 is a targeted device controller. In such a case, controller 305 performs specific control functions targeted to a specific device or component, such as a temperature control device or a power supply. For example, a temperature control device controller has the basic functionality to control a temperature control device. In other embodiments, controller 305 is a microprocessor. In such a case, controller 305 is programmable so that it can function to control more than one component of the device. In other cases, controller 305 is not a programmable microprocessor, but instead is a special purpose controller configured to control different components that perform different functions. While depicted as one component in FIG. 5, controller 305 may be made of many different components or integrated circuits.

Tip segment 205 is adapted to mate with or attach to limited reuse assembly 250. In the embodiment of FIG. 4, plunger interface 420 located on a bottom surface of plunger 415 is adapted to mate with mechanical linkage interface 545 located near a top surface of limited reuse assembly housing 255. In addition, tip interface connector 453 is adapted to connect with limited reuse assembly interface connector 553. When tip segment 205 is connected to limited reuse assembly 250 in this manner, actuator 515 and actuator shaft 510 are adapted to drive plunger 415 upward toward needle 210. In addition, an interface is formed between controller 305 and temperature control device 450. A signal can pass from controller 305 to temperature control device 450 through interface 535, limited reuse assembly interface connector 553, tip interface connector 453, and interface 530.

In operation, when tip segment 205 is connected to limited reuse assembly 250, controller 305 controls the operation of actuator 515. When actuator 515 is actuated, actuator shaft 510 is moved upward toward needle 210. In turn, mechanical linkage interface 545, which is mated with plunger interface 420, moves plunger 415 upward toward needle 210. A substance located in dispensing chamber 405 is then expelled through needle 210.

In addition, controller 305 controls the operation of temperature control device 450. Temperature control device 450 is adapted to heat and/or cool dispensing chamber housing 425 and its contents. Since dispensing chamber housing 425 is at least partially thermally conductive, heating or cooling dispensing chamber housing 425 heats or cools a substance located in dispensing chamber 405. Temperature information can be transferred from thermal sensor 460 through interface 530, tip interface connector 453, limited reuse assembly interface connector 553, and interface 535 back to controller 305. This temperature information can be used to control the operation of temperature control device 450. When temperature control device 450 is a heater, controller 305 controls the amount of current that is sent to temperature control device 450. The more current sent to temperature control device 450, the hotter it gets. In such a manner, controller 305 can use a feed back loop utilizing information from thermal sensor 460 to control the operation of temperature control device 450. Any suitable type of control algorithm, such as a proportional integral derivative (PID) algorithm, can be used to control the operation of temperature control device 450.

A substance to be delivered into an eye, typically a drug suspended in a phase transition compound, is located in dispensing chamber 405. In this manner, the drug and phase transition compound are contacted by the inner surface of dispensing chamber housing 425. The phase transition compound is in a solid or semi-solid state at lower temperatures and in a more liquid state at higher temperatures. Such a compound can be heated by the application of current to temperature control device 450 to a more liquid state and injected into the eye where it forms a bolus that erodes over time.

Likewise, a reverse gelation compound may be used. A reverse gelation compound is in a solid or semi-solid state at higher temperatures and in a more liquid state at lower temperatures. Such a compound can be cooled by temperature control device 450 to a more liquid state and injected into the eye where it forms a bolus that erodes over time. As such, temperature control device 450 may be a device that heats a substance in dispensing chamber 405 or a device that cools a substance in dispensing chamber 405 (or a combination of both). After being delivered into the eye, a phase transition compound or reverse gelation compound erodes over time providing a quantity of drug over an extended period of time. Using a phase transition compound or reverse gelation compound provides better drug dosage with fewer injections.

In one embodiment of the present invention, the substance located in dispensing chamber 405 is a drug that is preloaded into the dispensing chamber. In such a case, tip segment 205 is appropriate as a single use consumable product. Such a disposable product can be assembled at a factory with a dosage of a drug installed.

FIGS. 5A & 5B are cross section views of a tip segment for an ophthalmic medical device according to an embodiment of the present invention. In FIG. 5A, tip segment 205 includes dispensing chamber housing 425, tip segment housing 215, thermal sensor 460, needle 210, dispensing chamber 405, plunger 415, plunger interface 420, temperature control device 450, interface 530, tip interface connector 453, lever 610, living hinge 615, switch 620, interface 625, and contact 630.

In the embodiment of FIG. 5A, temperature control device 450 is activated to bring a substance in dispensing chamber 405 to within a proper temperature range. Thermal sensor 460 provides temperature information to controller 305 (not shown) to control temperature control device 450. After the substance has reached the proper temperature range, plunger 415 is actuated to deliver the substance through needle 210 and into an eye. Plunger 415 is extended and includes an integral shaft as shown.

Lever 610 is depressed to begin an injection. Lever 610 is ergonomically located near the tip end of disposable tip segment 205. In this manner, a doctor can control the insertion of needle 210 into an eye and depress lever 610 to inject a substance such as a drug. Hinge 615 is located near the end of disposable tip segment 205 that connects with a limited reuse assembly. Hinge 615 is connected to lever 610 such that an end user of the device, such as a doctor, can depress lever 610 so that it contacts switch 620.

Lever 610 rotates or translates downward so that it depresses a button on switch 620. While depicted as a switch with a button, switch 620 can be any type of switch. In another embodiment, the bottom surface of lever 610 is coated with a metallic substance. When lever 610 moves downward, the metallic substance contacts switch 620 and closes an electrical connection. In its simplest form, switch 610 may be two contacts that are closed by lever 610. Switch 620 is positioned such that lever 610 can actuate it.

An interface 625 connects switch 620 to contact 630. Contact 630, like tip interface connector 453, interfaces electrically with a limited reuse assembly. In this manner, the activation of switch 620 can be communicated to a limited reuse assembly. In this manner, depressing lever 610 actuates switch 620. The actuation of switch 620 is communicated via interface 625 and contact 630 to a limited reuse assembly. This actuation of switch 620 can then indicate the start of an injection procedure. A controller in a limited reuse assembly can detect the switch signal and begin an injection procedure.

Lever 610 and hinge 615 may be molded from a polymer or other similar material. In one embodiment of the present invention, lever 610 and hinge 615 may be molded from a first polymer or plastic material to form the structure of the hinge. A second polymer or plastic material (which may be of the same chemical composition as the first polymer or plastic material) may then be overmolded onto the first material to seal any gaps or cracks that were used to form lever 610 and hinge 615. For example, lever 610 may have gaps between it and the remainder of housing 215. A second material overmolded onto housing 215 may seal these gaps. In such a case lever 610 and hinge 615 may be integral with housing 215 (all of which may be formed in a plastic or polymer material—perhaps by an injection molding process).

FIG. 5B shows a larger view of lever 610, hinge 615, switch 620, and interface 625. Lever 610 has a hinge 615 located at one end as shown. Hinge 615 is connected to housing 215 such that the end of lever 610 can pivot about hinge 615. This end is designed to contact switch 620. While lever 610 is depicted as a having club-like form, any other suitable shape may be employed.

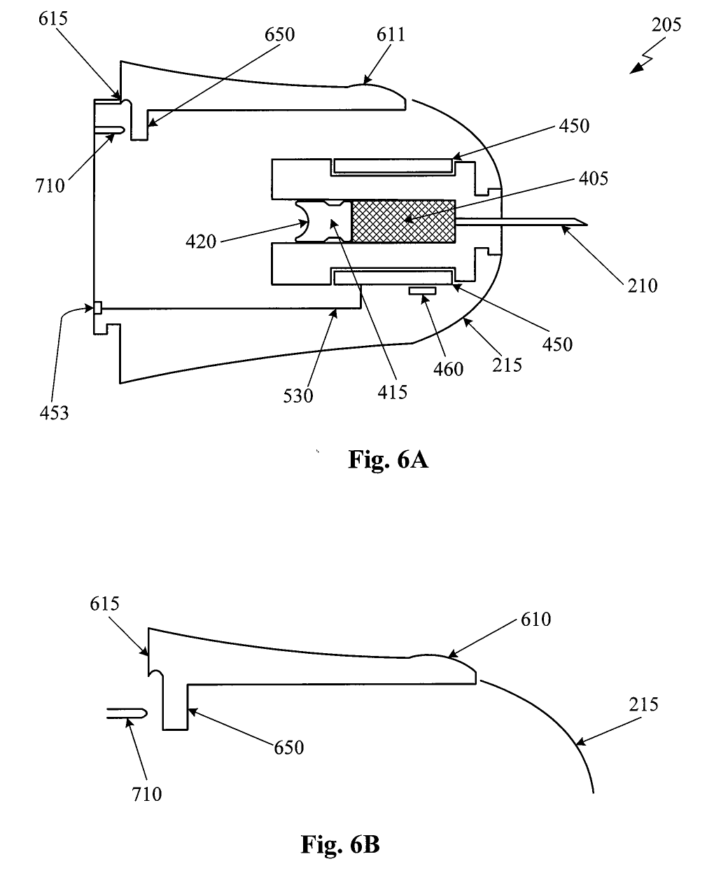

FIGS. 6A & 6B depict another arrangement for lever 610. In FIG. 6A, tip segment 205 includes dispensing chamber housing 425, tip segment housing 215, thermal sensor 460, needle 210, dispensing chamber 405, plunger 415, plunger interface 420, temperature control device 450, interface 530, tip interface connector 453, lever 611, hinge 615, and protrusion 650.

Lever 611 is capable of pivoting at hinge 615. As in FIG. 5, hinge 611 of FIG. 6 is capable of moving downward when depressed. Hinge 615 is the pivot point for lever 611. Protrusion 650 moves outward (in the direction away from the needle end of tip segment 205) when lever 611 is depressed. In this manner, lever 611 can be depressed and protrusion 650 will move. When protrusion 650 extends outward it closes contacts (710) located on the tip segment. In this manner, when tip segment 205 is attached to a limited reuse assembly, lever 611 can be depressed to engage protrusion 650 with contacts on the tip segment. Protrusion 650 closes the contacts resulting in a switching action.

As more clearly seen in FIG. 6B, when lever 611 is depressed, protrusion 650 contacts assembly 710. Assembly 710, in its simplest embodiment, includes two contacts. Protrusion 650 touches both contacts thus closing a switch. In other embodiments, assembly 710 may be a switch. In such a case, protrusion 650 operates the switch.

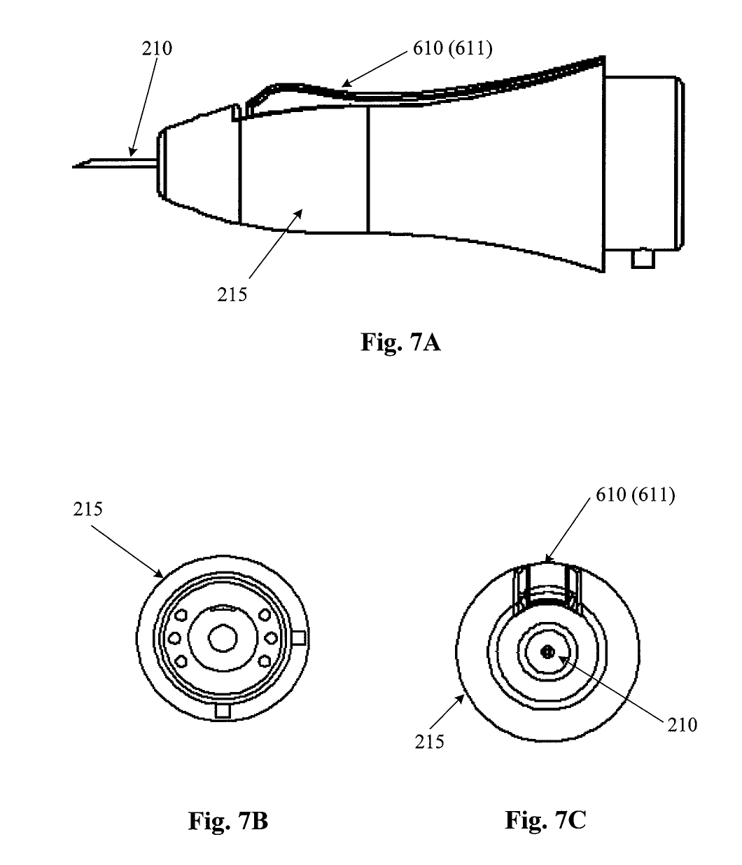

FIGS. 7A-7C show external views of the assemblies of FIGS. 5 and 6. FIG. 7A is a side view of a disposable tip segment according to the principles of the present invention. In FIG. 7A, lever 610 or 611 is integral with housing 215. As noted, both housing 215 and lever 610 or 611 are molded from a plastic, polymer or other like material. Needle 210 is also depicted. FIGS. 7B and 7C are end views of the tip segment of FIG. 7A.

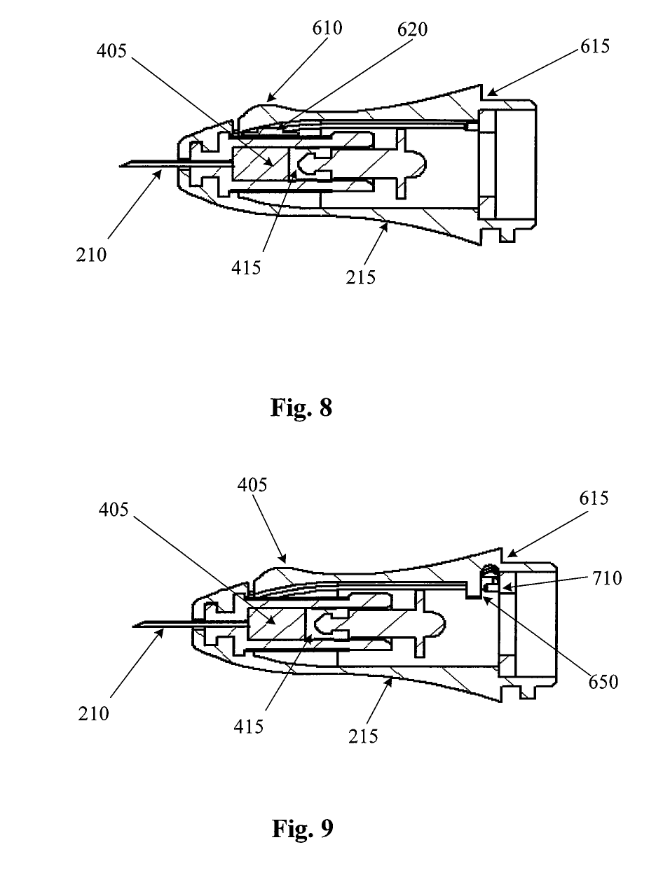

FIG. 8 is a cross section view of another embodiment of the tip segment of FIG. 5. In FIG. 8, tip segment 205 includes tip segment housing 215, needle 210, dispensing chamber 405, plunger 415, lever 610, living hinge 615, and switch 620.

FIG. 9 is a cross section view of another embodiment of the tip segment of FIG. 6. In FIG. 9, tip segment 205 includes tip segment housing 215, needle 210, dispensing chamber 405, plunger 415, lever 611, living hinge 615, and protrusion 650.

From the above, it may be appreciated that the present invention provides an improved system and method for delivering precise volumes of a substance. The present invention provides a two piece injection device with a consumable activation lever. The consumable activation lever is located on the disposable tip segment and implements a proper interface with the limited reuse assembly. The present invention is illustrated herein by example, and various modifications may be made by a person of ordinary skill in the art.

While described in terms of an ophthalmic injection device, the present invention is suitable for use in any type of injection device. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A disposable injection device assembly comprising:
a dispensing chamber housing coupled to a needle, the dispensing chamber housing having an inner surface and an outer surface, the inner surface partially defining a dispensing chamber for holding a quantity of a substance;
a plunger engaged with the inner surface of the dispensing chamber housing, the plunger capable of sliding in the dispensing chamber housing, the plunger fluidly sealed to the inner surface of the dispensing chamber housing;
a housing at least partially surrounding the dispensing chamber housing;
a lever located on the housing; and
a switch located adjacent to the lever;
wherein when the lever is depressed, the lever contacts the switch.

2. The assembly of claim 1 wherein the lever is integral with the housing.

3. The assembly of claim 1 wherein the lever is ergonomically located near the needle.

4. The assembly of claim 1 wherein the lever and housing are made of a polymeric material.

5. The assembly of claim 1 further comprising an overmolded layer of material on the housing and the lever.

6. The assembly of claim 1 further comprising a hinge located on one end of the lever, the hinge connecting the lever to the housing.

7. The assembly of claim 1 wherein the lever actuates the switch.

8. The assembly of claim 1 wherein the switch is located beneath the lever so that when the lever is depressed, the lever contacts and activates the switch.

9. The device of claim 1 wherein the substance is a drug for treating a condition of the eye.

10. A disposable injection device assembly comprising:
a dispensing chamber housing coupled to a needle, the dispensing chamber housing having an inner surface and an outer surface, the inner surface partially defining a dispensing chamber for holding a quantity of a substance;
a plunger engaged with the inner surface of the dispensing chamber housing, the plunger capable of sliding in the dispensing chamber housing, the plunger fluidly sealed to the inner surface of the dispensing chamber housing;
a housing at least partially surrounding the dispensing chamber housing; and
a lever located on the housing, the lever having a protrusion on one end;
wherein when the lever is depressed the protrusion is moved.

11. The assembly of claim 1 wherein the lever is integral with the housing.

12. The assembly of claim 1 wherein the lever is ergonomically located near the needle.

13. The assembly of claim 1 wherein the lever and housing are made of a polymeric material.

14. The assembly of claim 1 further comprising an overmolded layer of material on the housing and the lever.

15. The assembly of claim 1 further comprising a hinge located on one end of the lever, the hinge connecting the lever to the housing.

16. The assembly of claim 1 wherein the protrusion closes a set of contacts.

17. The assembly of claim 1 wherein a switch is located adjacent to the protrusion so that when the lever is depressed, the protrusion contacts and activates a switch.

18. The device of claim 1 wherein the substance is a drug for treating a condition of the eye.
